(19) United States
(12) Patent Application Publication
(10) Pub. No.: US 2024/0164676 A1
(43) Pub. Date: May 23, 2024

(54) INFORMATION PROCESSING SYSTEM

(71) Applicant: SONY GROUP CORPORATION, TOKYO (JP)

(72) Inventor: YASUHIDE HYODO, TOKYO (JP)

(21) Appl. No.: 18/551,681

(22) PCT Filed: Jan. 26, 2022
(86) PCT No.: PCT/JP2022/002932

§ 371 Date: Sep. 21, 2023

(30) Foreign Application Priority Data
Apr. 7, 2021 (JP) 2021-065169

(51) Int. Cl. A61B 5/16; A61B 5/00

(52) U.S. Cl. CPC A61B 5/165; A61B 5/7267

(57) ABSTRACT

An information processing system according to one aspect of the present disclosure includes an input information generation unit and a determination unit. The input information generation unit performs predetermined preprocessing on biological information of a target biological object acquired with a sensor, thereby generating input information with respect to each of a plurality of machine learning models. The determination unit determines an arousal of the target biological object on the basis of an estimation result obtained from each of the machine learning models by inputting the input information to each machine learning model.

### TECHNICAL FIELD

The present disclosure relates to an information processing system.

### BACKGROUND ART

A change in human emotion causes a physiological response such as brain waves, heartbeat, or perspiration, and it shows up on a body surface. By reading this physiological response as a biological signal with a sensor device, it becomes possible to estimate a human emotion. For example, by inputting, into a model formula found by machine learning, a feature amount such as a physiological index contributing to an emotional response that is obtained by performing predetermined signal processing on a read biological signal, it becomes possible to estimate a human emotion (for example, see PTLs 1 and 2).

### CITATION LIST

### Patent Literatures

PTL 1: International Publication No. WO2012/056546
PTL 2: Japanese Unexamined Patent Application Publication No. 2016-106689

### SUMMARY OF INVENTION

It is to be noted that an arousal baseline assumed at the time of model learning is not necessarily consistent with a user's arousal baseline. Because of this, there is an issue that it is not possible to accurately estimate an arousal. Therefore, it is desirable to provide an information processing system that makes it possible to accurately estimate the arousal.

An information processing system according to one aspect of the present disclosure includes an input information generation unit and a determination unit. The input information generation unit performs predetermined preprocessing on biological information of a target biological object acquired with a sensor, thereby generating input information with respect to each of a plurality of machine learning models. The determination unit determines an arousal of the target biological object on the basis of an estimation result obtained from each of the machine learning models by inputting the input information to the machine learning model.

In the information processing system according to one aspect of the present disclosure, the input information corresponding to the biological information of the target biological object acquired with the sensor is generated with respect to each of the plurality of machine learning models. Thus, input information that has taken into consideration a range of various parameters in each machine learning model may be generated. In this information processing system, a plurality of pieces of the input information generated for each machine learning model are inputted to their corresponding machine learning models, thereby the arousal of the target biological object is determined on the basis of an estimation result obtained from each of the machine learning models. Therefore, for example, it is possible to acquire an estimation result in accordance with the content and degree of the difference between the arousal baseline of each machine learning model and a user's arousal baseline from each machine learning model.

### BRIEF DESCRIPTION OF THE DRAWINGS

FIG. 1 is a diagram illustrating an example of a schematic configuration of an information processing system according to a first embodiment of the present disclosure.

FIGS. 2A to 2C are diagrams illustrating an example of teaching information (before normalization) used for learning of reference models in FIG. 1.

FIGS. 3A to 3C are diagrams illustrating an example of normalized teaching information.

FIG. 4 is a diagram illustrating an example of learning of the reference model.

FIG. 5 is a diagram illustrating an example of learning of the reference model.

FIG. 6 is a diagram illustrating an example of learning of the reference model.

FIG. 7 is a diagram illustrating an example of a procedure to acquire normalized information.

FIG. 8 is a diagram illustrating an example of a procedure to convert a normalization coefficient.

FIG. 9 is a diagram illustrating an example of a schematic configuration of an information processing system according to a second embodiment of the present disclosure.

FIGS. 10A to 10C are diagrams illustrating an example of standardized teaching information.

FIG. 11 is a diagram illustrating an example of learning of the reference model.

FIG. 12 is a diagram illustrating an example of learning of the reference model.

FIG. 13 is a diagram illustrating an example of learning of the reference model.

FIG. 14 is a diagram illustrating an example of a procedure to acquire standardized information.

FIG. 15 is a diagram illustrating an example of a procedure to convert a standardization coefficient.

FIG. 16 is a diagram illustrating a modification example of a schematic configuration of the information processing system in FIG. 1.

FIG. 17 is a diagram illustrating a modification example of a schematic configuration of the information processing system in FIG. 9.

### MODES FOR CARRYING OUT THE INVENTION

Embodiments of the present disclosure is described in detail below with reference to the drawings.

### 1. About Arousal

The arousal of a human has a lot to do with the human's ability to concentrate. When the human is concentrating, s/he takes a high degree of interest in an object of the concentration. Therefore, by knowing the arousal of a human, it becomes possible to estimate the degree of his/her objective interest (emotion). The arousal of the human is able to be derived on the basis of biological information obtained from, for example, a person (hereinafter, referred to as a “target biological object”) who is doing a job under office environment or doing yoga. The biological information that allows the arousal of the target biological object to be derived includes, for example, information regarding brain waves, perspiration, a pulse wave, an electrocardiogram, a blood flow, a skin temperature, a mimetic muscle potential, an electrooculogram, or a specific component contained in saliva. Embodiments of an information processing system that performs emotion estimation using biological information are described below.

### 2. First Embodiment

### Configuration

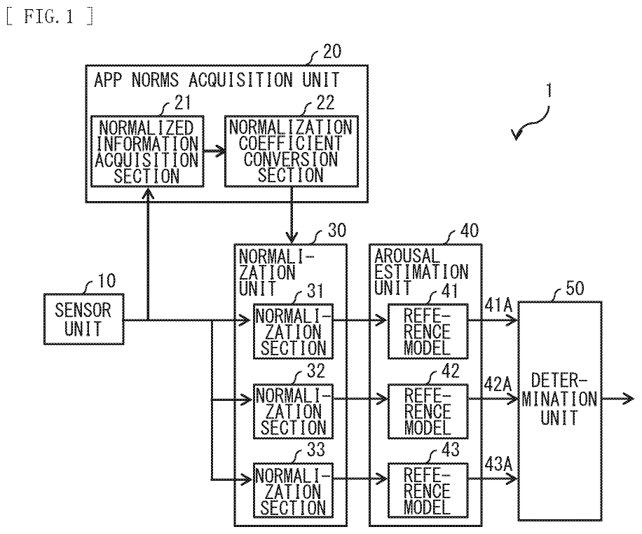

An information processing system 1 according to a first embodiment of the present disclosure is described. FIG. 1 illustrates an example of a schematic configuration of the information processing system 1. The information processing system 1 is a system that estimates an emotion of a target biological object on the basis of biological information obtained from the target biological object. In the present embodiment, the target biological object is a human. It is to be noted that in the information processing system 1, the target biological object is not limited to a human.

The information processing system 1 may be realized by a single electronic apparatus, or may be realized by a plurality of electronic apparatuses that are able to transmit and receive data to/from one another through a communication network. The information processing system 1 includes, for example, a sensor unit 10, an app norms acquisition unit 20, a normalization unit 30, an arousal estimation unit 40, and a determination unit 50. The arousal estimation unit 40 includes a plurality of machine learning models (for example, three reference models 41, 42, and 43) having different arousal baselines from one another. The machine learning models include, for example, a regression model or an identification model. That is, the information processing system 1 uses the plurality of machine learning models provided in the arousal estimation unit 40 to estimate the arousal of the target biological object.

The term arousal baseline means an average of before-normalization information of teaching information used for learning of each machine learning model. For example, the arousal baseline of the reference model 41 is lower than the arousal baselines of the reference models 42 and 43, and the arousal baseline of the reference model 42 is lower than the arousal baseline of the reference model 43.

The “before-normalization information of teaching information used for learning of each machine learning model” means, for example, feature amounts (not normalized feature amounts) generated on the basis of time-series data of biological information obtained by the sensor unit 10, etc. This feature amount may include a plurality of classes of different arousal levels from one another. This means, for example, that while the target biological object is at a predetermined arousal level under a certain environment, the arousal level varies depending on the times and circumstances. It is to be noted that a target biological object at the time of generation of teaching information is generally different from a target biological object whose emotion is to be estimated by the information processing system 1.

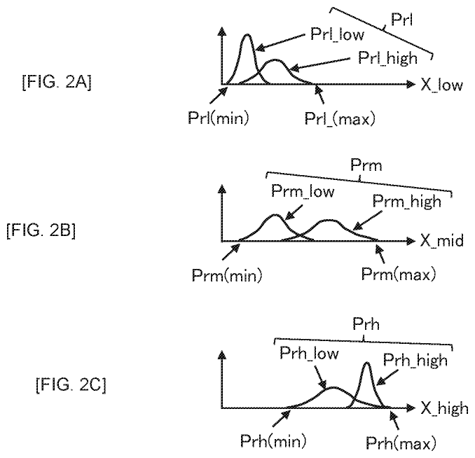

For example, as illustrated in FIG. 2(A), a feature amount X_low obtained when the arousal level of a target biological object is relatively low includes two classes of different arousal levels from each other: Prl_low and Prl_high. It is to be noted that in FIG. 2(A), data including the two classes, Prl_low and Prl_high, is denoted by Prl. Furthermore, for example, as illustrated in FIG. 2(C), a feature amount X_high obtained when the arousal level of the target biological object is relatively high includes two classes of different arousal levels from each other: Prh_low and Prh_high. It is to be noted that in FIG. 2(C), data including the two classes, Prh_low and Prh_high, is denoted by Prh. Moreover, for example, as illustrated in FIG. 2(B), a feature amount X_mid obtained when the arousal level of the target biological object is in between the arousal level in FIG. 2(A) and the arousal level in FIG. 2(C) includes two classes of different arousal levels from each other: Prm_low and Prm_high. It is to be noted that in FIG. 2(B), data including the two classes, Prm_low and Prm_high, is denoted by Prm.

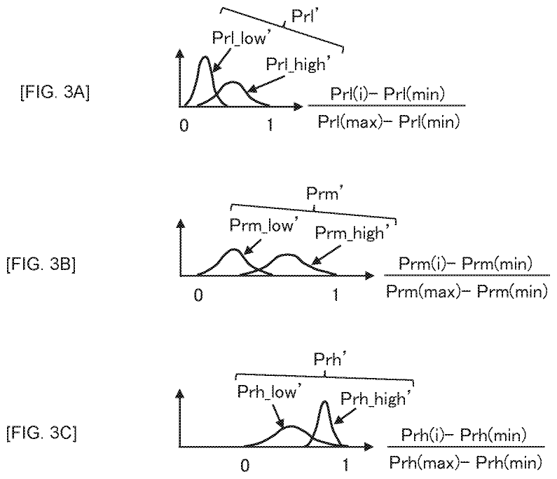

FIG. 3(A) illustrates an example of data Prl′ (normalized information) obtained by normalizing the data Prl illustrated in FIG. 2(A). In FIG. 3(A), the horizontal axis represents (Prl(i)−Prl(min))/(Prl(max)−Prl(min)), where Prl(min) denotes the minimum value of the feature amount of Prl, Prl(max) denotes the maximum value of the feature amount of Prl, and i denotes a variable that may have a value within a range of 0≤i≤ 1. In FIG. 3(A), Prl_low′ is data (normalized information) corresponding to Prl_low and obtained by normalization of the data Prl. In FIG. 3(A), Prl_high′ is data (normalized information) corresponding to Prl_high and obtained by normalization of the data Prl.

FIG. 3(B) illustrates an example of data Prm′ (normalized information) obtained by normalizing the data Prm illustrated in FIG. 2(B). In FIG. 3(B), the horizontal axis represents (Prm(i)−Prm(min))/(Prm(max)−Prm(min)), where Prm(min) denotes the minimum value of the feature amount of Prm, and Prm(max) denotes the maximum value of the feature amount of Prm. In FIG. 3(B), Prm_low′ is data (normalized information) corresponding to Prm_low and obtained by normalization of the data Prm. In FIG. 3(B), Prm_high′ is data (normalized information) corresponding to Prm_high and obtained by normalization of the data Prm.

FIG. 3(C) illustrates an example of data Prh′ (normalized information) obtained by normalizing the data Prh illustrated in FIG. 2(C). In FIG. 3(C), the horizontal axis represents (Prh(i)−Prh(min))/(Prh(max)−Prh(min)), where Prh(min) denotes the minimum value of the feature amount of Prh, and Prh(max) denotes the maximum value of the feature amount of Prh. In FIG. 3(C), Prh_low′ is data (normalized information) corresponding to Prh_low and obtained by normalization of the data Prh. In FIG. 3(C), Prh_high′ is data (normalized information) corresponding to Prh_high and obtained by normalization of the data Prh.

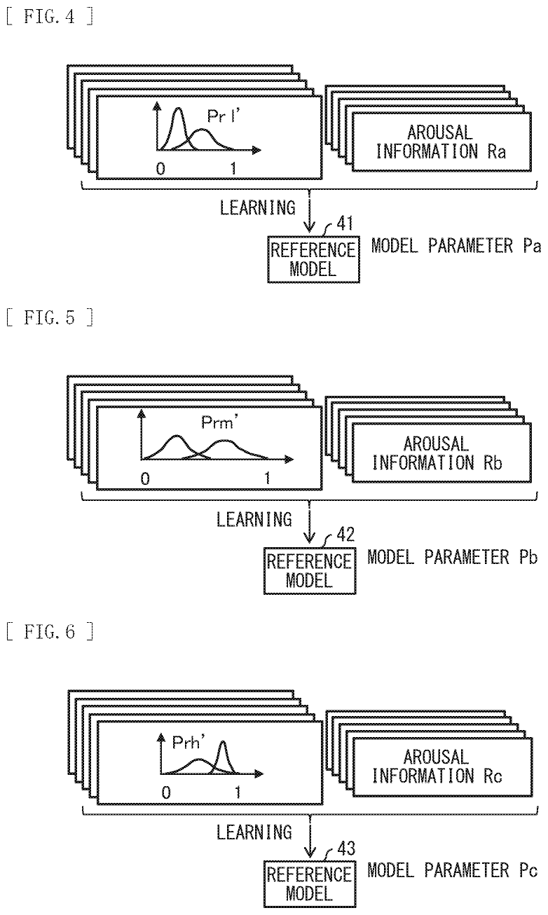

The reference models 41, 42, and 43 are models generated by using respective pieces of data acquired under environments of different arousal levels from one another. The reference model 41 is a model generated by using the normalized information of the feature amount of the data Prl obtained on a relatively low arousal baseline and arousal information Ra (see FIG. 4). A model parameter Pa obtained through this learning is stored in the arousal estimation unit 40. The reference model 43 is a model generated by using the normalized information of the feature amount of the data Prh obtained on a relatively high arousal baseline and arousal information Rc (see FIG. 6). A model parameter Pc obtained through this learning is stored in the arousal estimation unit 40. The reference model 42 is a model generated by using the normalized information of the feature amount of the data Prm obtained on an arousal baseline between the arousal baseline of the reference model 41 and the arousal baseline of the reference model 43 and arousal information Rb (see FIG. 5). A model parameter Pb obtained through this learning is stored in the arousal estimation unit 40.

The sensor unit 10 may be, for example, a sensor of a contact type that has contact with a target biological object, or may be a non-contact sensor that has no contact with a target biological object. The sensor unit 10 is a sensor that acquires, for example, of pieces of information regarding brain waves, perspiration, a pulse wave, an electrocardiogram, a blood flow, a skin temperature, a mimetic muscle potential, an electrooculogram, and a specific component contained in saliva, one or more pieces of information (biological information). The sensor unit 10 outputs the acquired biological information to the app norms acquisition unit 20 and the normalization unit 30.

The app norms acquisition unit 20 optimizes conditions for normalization in accordance with the action state of the target biological object. The app norms acquisition unit 20 includes a normalized information acquisition section 21 and a normalization coefficient conversion section 22.

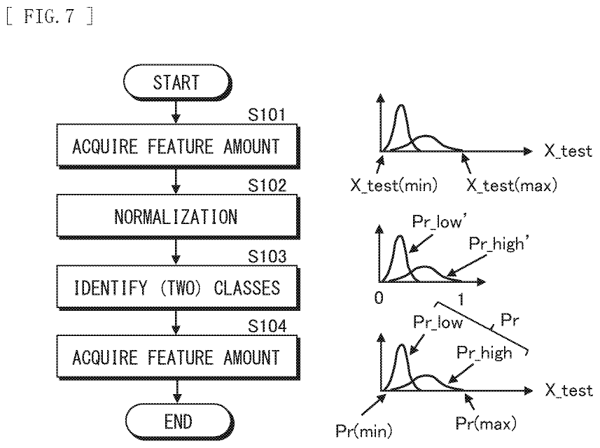

The normalized information acquisition section 21 acquires a feature amount X_test on the basis of time-series data of biological information (hereinafter, “biological information 10A”) acquired by using the sensor unit 10 before time-series data of biological information (hereinafter, “biological information 10B”) used for generation of information inputted from the normalization unit 30 to the arousal estimation unit 40 is acquired (FIG. 7, step S101). The normalized information acquisition section 21 normalizes the acquired feature amount X_test into X_test(max) and X_test(min) (FIG. 7, step S102). The term X_test(max) denotes the maximum value of the acquired feature amount X_test. The term X_test(min) denotes the minimum value of the acquired feature amount X_test. The normalized information acquisition section 21 identifies a plurality of classes of different arousal levels from one another in a feature amount X_test′ obtained by the normalization, thereby acquiring feature amounts of the plurality of classes. For example, the normalized information acquisition section 21 identifies Pr_low′ and Pr_high′, two classes of different arousal levels from each other, in the feature amount X_test′ obtained by the normalization (FIG. 7, step S103). For example, the normalized information acquisition section 21 acquires Pr_low and Pr_high, before-normalization classes corresponding to the classes Pr_low′ and Pr_high′ obtained as a result of the identification (FIG. 7, step S104). The normalized information acquisition section 21 outputs a feature amount of data including the acquired plurality of classes (for example, a feature amount of data Pr including the two classes Pr_low and Pr_high) to the normalization coefficient conversion section 22.

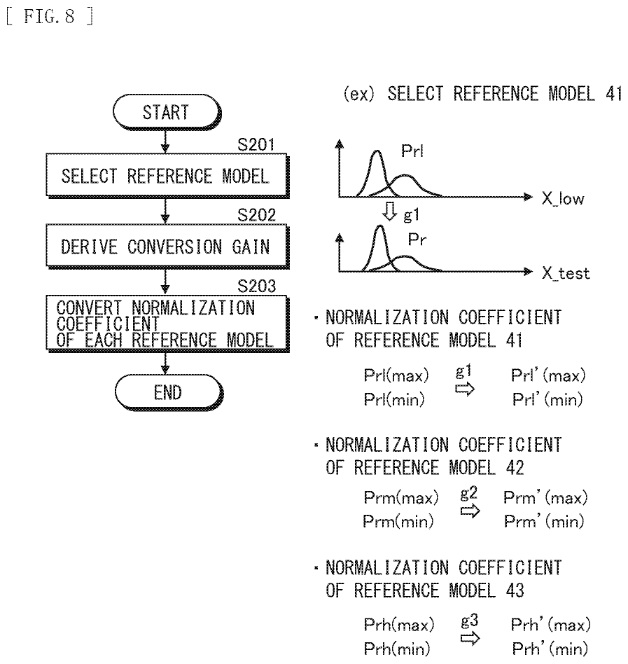

The normalization coefficient conversion section 22 selects, of the plurality of machine learning models included in the arousal estimation unit 40, one machine learning model (FIG. 8, step S201). The normalization coefficient conversion section 22 selects, for example, the reference model 41. For example, the normalization coefficient conversion section 22 selects, of the plurality of machine learning models included in the arousal estimation unit 40, a machine learning model having an arousal baseline closest to an arousal baseline of the feature amount X_test obtained on the basis of the biological information 10A. One reason for this is to accurately perform mapping to be described later.

On the basis of the feature amount inputted from the normalized information acquisition section 21 and a feature amount used at the time of learning of the selected machine learning model, the normalization coefficient conversion section 22 converts a normalization coefficient of this machine learning model. For example, the normalization coefficient conversion section 22 derives, from the feature amount used at the time of learning of the selected machine learning model, a conversion gain with which mapping into the feature amount inputted from the normalized information acquisition section 21 is performed (FIG. 8, step S202). For example, the normalization coefficient conversion section 22 derives, from the feature amount of the data Prl used at the time of learning of the selected reference model 41, a conversion gain g1 with which mapping into the feature amount of the data Pr inputted from the normalized information acquisition section 21 is performed.

The normalization coefficient conversion section 22 converts a normalization coefficient of each machine learning model (FIG. 8, step S203). For example, the normalization coefficient conversion section 22 converts normalization coefficients Prl(max) and Prl(min) of the selected reference model 41 into normalization coefficients Prl′(max) and Prl′(min) with the conversion gain g1.

A mathematical function α that defines a correlation between the feature amount of the data Prl used at the time of learning of the reference model 41 and the feature amount of the data Prm used at the time of learning of the reference model 42 is assumed to have been stored in the normalization coefficient conversion section 22. Furthermore, a mathematical function β that defines a correlation between the feature amount of the data Prm used at the time of learning of the reference model 42 and the feature amount of the data Prh used at the time of learning of the reference model 43 is assumed to have been stored in the normalization coefficient conversion section 22.

For example, with the conversion gain g1 and the mathematical function α, the normalization coefficient conversion section 22 converts normalization coefficients Prm(max) and Prm(min) into normalization coefficients Prm′(max) and Prm′(min). It is to be noted that in FIG. 8, the conversion gain g1 and the mathematical function α are represented by a conversion gain g2. Furthermore, for example, with the conversion gain g1 and the mathematical functions α and β, the normalization coefficient conversion section 22 converts normalization coefficients Prh(max) and Prh(min) into normalization coefficients Prh′(max) and Prh′(min). It is to be noted that in FIG. 8, the conversion gain g1 and the mathematical functions α and β are represented by a conversion gain g3.

The normalization coefficient conversion section 22 outputs the derived conversion gains g1, g2, and g3 to the normalization unit 30.

The normalization unit 30 includes a plurality of normalization sections (for example, three normalization sections 31, 32, and 33); one normalization unit is provided for each of the machine learning models (for example, the reference models 41, 42, and 43) included in the arousal estimation unit 40. The normalization section 31 is provided to correspond to the reference model 41. The normalization section 32 is provided to correspond to the reference model 42. The normalization section 33 is provided to correspond to the reference model 43.

The normalization unit 30 acquires a feature amount X_test on the basis of the biological information 10B obtained by using the sensor unit 10. The normalization unit 30 normalizes the acquired feature amount X_test into X_test(max) and X_test(min). The normalization unit 30 identifies Pr_low′ and Pr_high′, two classes of different arousal levels from each other, in the normalized feature amount X_test′. The normalization unit 30 acquires Pr_low and Pr_high, before-normalization classes corresponding to the classes Pr_low′ and Pr_high′ obtained as a result of the identification. The normalization unit 30 inputs a feature amount of data Pr including the two classes Pr_low and Pr_high to the normalization sections 31, 32, and 33. The normalization sections 31, 32, and 33 perform predetermined normalization on the inputted feature amount of the data Pr.

The normalization section 31 converts the feature amount of the data Pr obtained using the biological information 10B with the conversion gain g1 inputted from the normalization coefficient conversion section 22, thereby acquiring a feature amount of data Pra. The normalization section 31 normalizes the acquired feature amount of the data Pra into Pra(max) and Pra(min), and outputs obtained data Pra′ to the reference model 41. The term Pra(max) denotes the maximum value of the feature amount of Pra. The term Pra(min) denotes the minimum value of the feature amount of Pra.

The normalization section 32 converts the feature amount of the data Pr obtained using the biological information 10B with the conversion gain g2 inputted from the normalization coefficient conversion section 22, thereby acquiring a feature amount of data Prb. The normalization section 32 normalizes the acquired feature amount of the data Prb into Prb(max) and Prb(min), and outputs obtained data Prb′ to the reference model 42. The term Prb(max) denotes the maximum value of the feature amount of Prb. The term Prb(min) denotes the minimum value of the feature amount of Prb.

The normalization section 33 converts the feature amount of the data Pr obtained using the biological information 10B with the conversion gain g3 inputted from the normalization coefficient conversion section 22, thereby acquiring a feature amount of data Prc. The normalization section 33 normalizes the acquired feature amount of the data Prc into Prc(max) and Prc(min), and outputs obtained data Prc′ to the reference model 43. The term Prc(max) denotes the maximum value of the feature amount of Prc. The term Prc(min) denotes the minimum value of the feature amount of Prc.

When having received the normalized data (the data Pra′) from the normalization section 31, the reference model 41 outputs arousal information 41A corresponding to the feature amount of the input data Pra′. When having received the normalized data (the data Prb′) from the normalization section 32, the reference model 42 outputs arousal information 42A corresponding to the feature amount of the received data Prb′. When having received the normalized data (the data Prc′) from the normalization section 33, the reference model 43 outputs arousal information 43A corresponding to the feature amount of the received data Prc′.

The determination unit 50 determines the arousal of the target biological object on the basis of an estimation result (the arousal information 41A, 42A, and 43A) inputted from the arousal estimation unit 40. The determination unit 50 determines the arousal of the target biological object, for example, by a majority vote based on the estimation result (the arousal information 41A, 42A, and 43A). For example, it is assumed that the arousal information 41A is information that means the arousal is high, the arousal information 42A is information that means the arousal is low, and the arousal information 43A is information that means the arousal is low. In this case, a result is 2 votes for arousal=low and 1 vote for arousal=high; therefore, a determination result that means the arousal is low is generated in accordance with the majority vote. For example, the determination unit 50 may further estimate an emotion of the target biological object on the basis of the generated determination result. It is to be noted that the determination unit 50 may use a method other than a majority vote to determine the arousal of the target biological object.

### Effects

In the information processing system 1 according to the present embodiment, input information corresponding to biological information 10B of a target biological object obtained by the sensor unit 10 is generated for each of a plurality of machine learning models having different arousal baselines from one another. For example, as the input information, a feature amount of data Pra′ is generated for the reference model 41. Furthermore, for example, as the input information, a feature amount of data Prb′ is generated for the reference model 42. Moreover, for example, as the input information, a feature amount of data Prc′ is generated for the reference model 43. Thus, the input information that has taken into consideration a range of various parameters in each machine learning model may be generated. In this information processing system 1, a plurality of pieces of the input information generated for each machine learning model are inputted to their corresponding machine learning models, thereby an emotion of the target biological object is determined on the basis of an estimation result obtained from each of the machine learning models. Therefore, an estimation result in accordance with the content and degree of the difference between the arousal baseline of each machine learning model and a user's arousal baseline is obtained from each of the machine learning models. Consequently, even in a case where the arousal baseline assumed at the time of model learning is not consistent with the user's arousal baseline, it is possible to accurately determine an emotion of the target biological object.

Furthermore, in the information processing system 1 according to the present embodiment, a feature amount is acquired on the basis of biological information 10A of the target biological object acquired by using the sensor unit 10 before the biological information 10B used for generation of the input information is acquired. In this information processing system 1, further, on the basis of the acquired feature amount and a feature amount used at the time of learning of one reference model (a first machine learning model) in the plurality of machine learning models, a normalization coefficient of the first machine learning model is converted. Therefore, an estimation result in accordance with the content and degree of the difference between the arousal baseline of each machine learning model and the user's arousal baseline is obtained from each of the machine learning models. Consequently, even in a case where the arousal baseline assumed at the time of model learning is not consistent with the user's arousal baseline, it is possible to accurately determine an emotion of the target biological object.

Moreover, in the information processing system 1 according to the present embodiment, from the feature amount used at the time of learning of one machine learning model (the first machine learning model) in the plurality of machine learning models, a conversion gain (a first conversion gain) with which mapping into the feature amount acquired on the basis of the biological information 10A is performed is derived. Then, by using the derived conversion gain, the normalization coefficient of the first machine learning model is converted. Consequently, even in a case where the arousal baseline assumed at the time of model learning is not consistent with the user's arousal baseline, it is possible to accurately determine an emotion of the target biological object.

Furthermore, in the information processing system 1 according to the present embodiment, by using a second conversion gain that describes a correlation between the feature amount used at the time of learning of the first machine learning model and a feature amount used at the time of learning of a second machine learning model different from the first machine learning model and the first conversion gain, a normalization coefficient of the second machine learning model is converted. Consequently, even in a case where the arousal baseline assumed at the time of model learning is not consistent with the user's arousal baseline, it is possible to accurately determine an emotion of the target biological object.

### 3. Second Embodiment

### Configuration

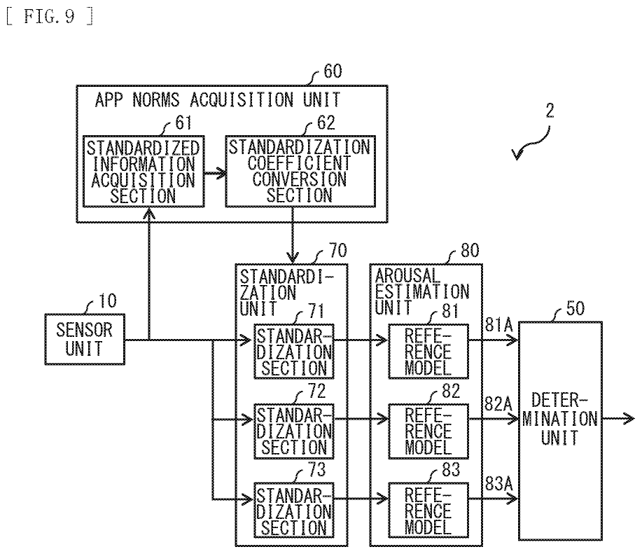

Subsequently, an information processing system 2 according to a second embodiment of the present disclosure is described. FIG. 9 illustrates an example of a schematic configuration of the information processing system 2. The information processing system 2 is a system that estimates an emotion of a target biological object on the basis of biological information obtained from the target biological object. In the present embodiment, the target biological object is a human. It is to be noted that in the information processing system 2, the target biological object is not limited to a human.

The information processing system 2 may be realized by a single electronic apparatus, or may be realized by a plurality of electronic apparatuses that are able to transmit and receive data to/from one another through a communication network. The information processing system 2 includes, for example, the sensor unit 10, an app norms acquisition unit 60, a standardization unit 70, an arousal estimation unit 80, and the determination unit 50. The arousal estimation unit 80 includes a plurality of machine learning models (for example, three reference models 81, 82, and 83) having different arousal baselines from one another. The machine learning models include, for example, a regression model or an identification model. That is, the information processing system 2 uses the plurality of machine learning models provided in the arousal estimation unit 80 to estimate the arousal of the target biological object.

The term arousal baseline means an average of before-standardization information of teaching information used for learning of each machine learning model. For example, the arousal baseline of the reference model 81 is lower than the arousal baselines of the reference models 82 and 83, and the arousal baseline of the reference model 82 is lower than the arousal baseline of the reference model 83.

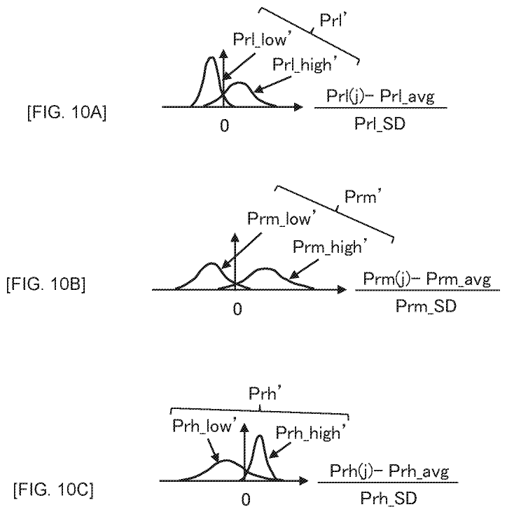

FIG. 10(A) illustrates an example of data Prl′ (standardized information) obtained by standardizing the data Prl illustrated in FIG. 2(A). In FIG. 10(A), the horizontal axis represents (Prl(j)−Prl_avg)/Prl_SD, where Prl_avg denotes the average of a feature amount of the data Prl, and Prl_SD denotes the variance of the feature amount of the data Prl. In FIG. 10(A), Prl_low′ is data (standardized information) corresponding to Prl_low and obtained by standardization of the data Prl. In FIG. 10(A), Prl_high′ is data (standardized information) corresponding to Prl_high and obtained by standardization of the data Prl.

FIG. 10(B) illustrates an example of data Prm′ (standardized information) obtained by standardizing the data Prm illustrated in FIG. 2(B). In FIG. 10(B), the horizontal axis represents (Prm(j)_avg)/Prm_SD, where Prm_avg denotes the average of a feature amount of the data Prm, and Prm_SD denotes the variance of the feature amount of the data Prm. In FIG. 10(B), Prm_low′ is data (standardized information) corresponding to Prm_low and obtained by standardization of the data Prm. In FIG. 10(B), Prm_high′ is data (standardized information) corresponding to Prm_high and obtained by standardization of the data Prm.

FIG. 10(C) illustrates an example of data Prh′ (standardized information) obtained by standardizing the data Prh illustrated in FIG. 2(C). In FIG. 10(C), the horizontal axis represents (Prh(j)−Prh_avg)/Prh_SD, where Prh_avg denotes the average of a feature amount of the data Prh, and Prh_SD denotes the variance of the feature amount of the data Prh. In FIG. 10(C), Prh_low′ is data (standardized information) corresponding to Prh_low and obtained by standardization of the data Prh. In FIG. 10(C), Prh_high′ is data (standardized information) corresponding to Prh_high and obtained by standardization of the data Prh.

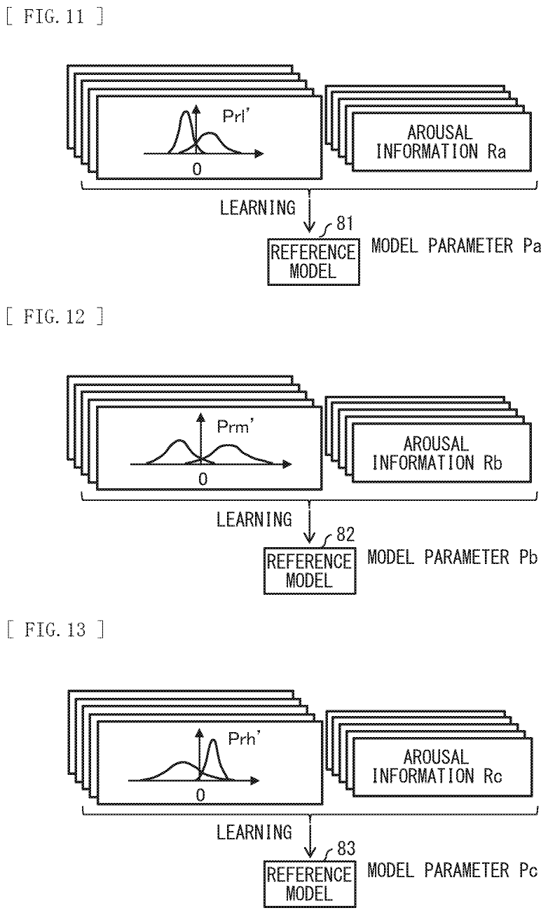

The reference models 1, 82, and 83 are models generated by using respective pieces of data acquired under environments of different arousal levels from one another. The reference model 81 is a model that has learned, as teaching information, the standardized information of the feature amount of the data Prl obtained on a relatively low arousal baseline and arousal information Ra (see FIG. 11). A model parameter Pa obtained through this learning is stored in the arousal estimation unit 80. The reference model 83 is a model that has learned, as teaching information, the standardized information of the feature amount of the data Prh obtained on a relatively high arousal baseline and arousal information Rc (see FIG. 12). A model parameter Pc obtained through this learning is stored in the arousal estimation unit 80. The reference model 82 is a model that has learned, as teaching information, the standardized information of the feature amount of the data Prm obtained on an arousal baseline between the arousal baseline of the reference model 81 and the arousal baseline of the reference model 83 and arousal information Rb (see FIG. 13). A model parameter Pb obtained through this learning is stored in the arousal estimation unit 80.

The sensor unit 10 may be, for example, a sensor of a contact type that has contact with a target biological object, or may be a non-contact sensor that has no contact with a target biological object. The sensor unit 10 is a sensor that acquires, for example, of pieces of information regarding brain waves, perspiration, a pulse wave, an electrocardiogram, a blood flow, a skin temperature, a mimetic muscle potential, an electrooculogram, and a specific component contained in saliva, one or more pieces of information (biological information). The sensor unit 10 outputs the acquired biological information to the app norms acquisition unit 60 and the standardization unit 70.

The app norms acquisition unit 60 optimizes conditions for standardization in accordance with the action state of the target biological object. The app norms acquisition unit 60 includes a standardized information acquisition section 61 and a standardization coefficient conversion section 62.

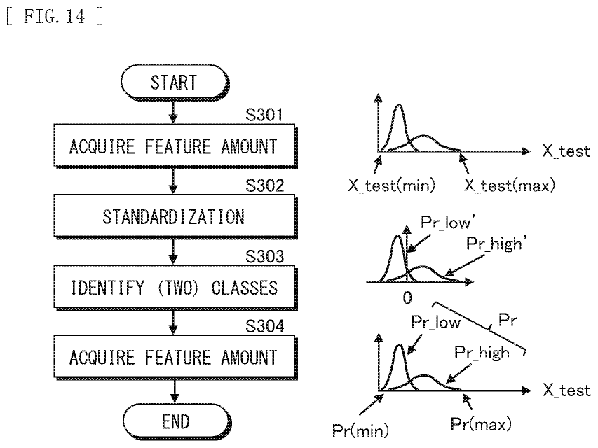

The standardized information acquisition section 61 acquires a feature amount X_test on the basis of biological information (hereinafter, “biological information 10A”) acquired by using the sensor unit 10 before biological information (hereinafter, “biological information 10B”) used for generation of information inputted from the standardization unit 70 to the arousal estimation unit 80 is acquired (FIG. 14, step S301). The standardized information acquisition section 61 standardizes the acquired feature amount X_test into X_test_avg and X_test_SD (FIG. 14, step S302). The term X_test_avg denotes the average of X_test. The term X_test_SD denotes the variance of X_test. The standardized information acquisition section 61 identifies a plurality of classes of different arousal levels from one another in the acquired feature amount X_test, thereby acquiring feature amounts of the plurality of classes. For example, the standardized information acquisition section 61 identifies Pr_low′ and Pr_high′, two classes of different arousal levels from each other, in the standardized feature amount X_test (FIG. 14, step S303). For example, the standardized information acquisition section 61 acquires Pr_low and Pr_high that are respective before-standardization classes corresponding to the classes Pr_low′ and Pr_high′ obtained as a result of the identification (FIG. 14, step S304). The standardized information acquisition section 61 outputs a feature amount of data including the acquired plurality of classes (for example, a feature amount of data Pr including the two classes Pr_low and Pr_high) to the standardization coefficient conversion section 62.

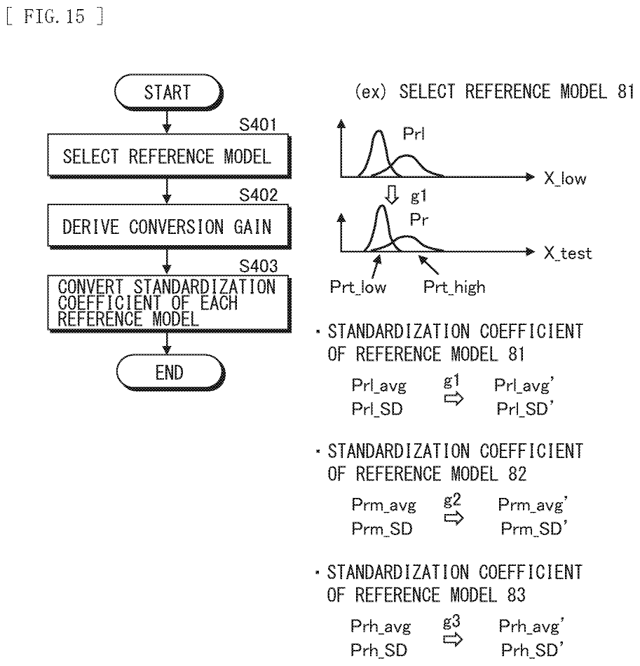

The standardization coefficient conversion section 62 selects, of the plurality of machine learning models included in the arousal estimation unit 80, one machine learning model (FIG. 15, step S401). The standardization coefficient conversion section 62 selects, for example, the reference model 81. For example, the standardization coefficient conversion section 62 selects, of the plurality of machine learning models included in the arousal estimation unit 80, a machine learning model having an arousal baseline closest to an arousal baseline of the feature amount X_test obtained on the basis of the biological information 10A. One reason for this is to accurately perform mapping to be described later.

On the basis of the feature amount inputted from the standardized information acquisition section 61 and a feature amount used at the time of learning of the selected machine learning model, the standardization coefficient conversion section 62 converts a standardization coefficient of this machine learning model. For example, the standardization coefficient conversion section 62 derives, from the feature amount used at the time of learning of the selected machine learning model, a conversion gain with which mapping into the feature amount inputted from the standardized information acquisition section 61 is performed (FIG. 15, step S402). For example, the standardization coefficient conversion section 62 derives, from the feature amount of data Prl used at the time of learning of the selected reference model 81, a conversion gain g1 with which mapping into the feature amount of the data Pr inputted from the standardized information acquisition section 61 is performed.

The standardization coefficient conversion section 62 converts a standardization coefficient of each machine learning model (FIG. 15, step S403). For example, the standardization coefficient conversion section 62 converts standardization coefficients Prl_avg and Prl_SD of the selected reference model 81 into standardization coefficients Prl_avg′ and Prl_SD′ with the conversion gain g1.

A mathematical function α that defines a correlation between the feature amount of the data Prl used at the time of learning of the reference model 81 and a feature amount of data Prm used at the time of learning of the reference model 82 is assumed to have been stored in the standardization coefficient conversion section 62. Furthermore, a mathematical function β that defines a correlation between the feature amount of the data Prm used at the time of learning of the reference model 82 and a feature amount of data Prh used at the time of learning of the reference model 83 is assumed to have been stored in the standardization coefficient conversion section 62.

For example, with the conversion gain g1 and the mathematical function α, the standardization coefficient conversion section 62 converts standardization coefficients Prm_avg and Prm_SD into standardization coefficients Prm_avg′ and Prm_SD′. It is to be noted that in FIG. 15, the conversion gain g1 and the mathematical function α are represented by a conversion gain g2. Furthermore, for example, with the conversion gain g1 and the mathematical functions α and β, the standardization coefficient conversion section 62 converts standardization coefficients Prh_avg and Prh_SD into standardization coefficients Prh_avg′ and Prh_SD′. It is to be noted that in FIG. 15, the conversion gain g1 and the mathematical functions α and β are represented by a conversion gain g3.

The standardization coefficient conversion section 62 outputs the derived conversion gains g1, g2, and g3 to the standardization unit 70.

The standardization unit 70 includes a plurality of standardization sections (for example, three standardization sections 71, 72, and 73); one standardization unit is provided for each of the machine learning models (for example, the reference models 81, 82, and 83) included in the arousal estimation unit 80. The standardization section 71 is provided to correspond to the reference model 81. The standardization section 72 is provided to correspond to the reference model 82. The standardization section 73 is provided to correspond to the reference model 83.

The standardization unit 70 acquires a feature amount X_test on the basis of the biological information 10B obtained by using the sensor unit 10. The standardization unit 70 standardizes the acquired feature amount X_test into X_test_avg and X_test_SD. The standardization unit 70 identifies Pr_low′ and Pr_high′, two classes of different arousal levels from each other, in the standardized feature amount X_test′. The standardization unit 70 acquires Pr_low and Pr_high that are respective before-standardization classes corresponding to the classes Pr_low′ and Pr_high′ obtained as a result of the identification. The standardization unit 70 inputs a feature amount of data Pr including the two classes Pr_low and Pr_high to the standardization sections 71, 72, and 73. The standardization sections 71, 72, and 73 perform predetermined standardization on the input feature amount of the inputted data Pr.

The standardization section 71 converts the feature amount of the data Pr obtained using the biological information 10B with the conversion gain g1 inputted from the standardization coefficient conversion section 62, thereby acquiring a feature amount of data Pra. The standardization section 71 standardizes the acquired feature amount of the data Pra into Pra_avg and Pra_SD, and outputs obtained data Pra′ to the reference model 81. The term Pra_avg denotes the average of the feature amount of the data Pra. The term Pra_SD denotes the variance of the feature amount of the data Pra.

The standardization section 72 converts the feature amount of the data Pr obtained using the biological information 10B with the conversion gain g2 inputted from the standardization coefficient conversion section 62, thereby acquiring a feature amount of data Prb. The standardization section 72 standardizes the acquired feature amount of the data Prb into Prb_avg and Prb_SD, and outputs a feature amount of obtained data Prb′ to the reference model 82. The term Prb_avg denotes the average of the feature amount of the data Prb. The term Prb_SD denotes the variance of the feature amount of the data Prb.

The standardization section 73 converts the feature amount of the data Pr obtained using the biological information 10B with the conversion gain g3 inputted from the standardization coefficient conversion section 62, thereby acquiring a feature amount of data Prc. The standardization section 73 standardizes the acquired feature amount of the data Prc into Prc_avg and Prc_SD, and outputs a feature amount of obtained data Prc′ to the reference model 82. The term Prc_avg denotes the average of the data Prc. The term Prc_SD denotes the variance of the feature amount of the data Prc.

When having received the normalized data (the data Pra′) from the standardization section 71, the reference model 81 outputs arousal information 81A corresponding to the feature amount of the input data Pra′. When having received the normalized data (the data Prb′) from the standardization section 72, the reference model 82 outputs arousal information 82A corresponding to the feature amount of the input data Prb′. When having received the normalized data (the data Prc′) from the standardization section 73, the reference model 83 outputs arousal information 83A corresponding to the feature amount of the inputted data Prc′.

The determination unit 50 determines the arousal of the target biological object on the basis of an estimation result (the arousal information 81A, 82A, and 83A) inputted from the arousal estimation unit 80. The determination unit 50 determines the arousal of the target biological object, for example, by a majority vote based on the estimation result (the arousal information 81A, 82A, and 83A). For example, it is assumed that the arousal information 81A is information that means the arousal is high, the arousal information 82A is information that means the arousal is low, and the arousal information 83A is information that means the arousal is low. In this case, a result is 2 votes for arousal=low and 1 vote for arousal-high; therefore, a determination result that means the arousal is low is generated in accordance with the majority vote. For example, the determination unit 50 may further estimate an emotion of the target biological object on the basis of the generated determination result. It is to be noted that the determination unit 50 may use a method other than a majority vote to determine the arousal of the target biological object.

### Effects

In the information processing system 2 according to the present embodiment, input information corresponding to biological information 10B of a target biological object obtained by using the sensor unit 10 is generated for each of a plurality of machine learning models having different arousal baselines from one another. For example, as the input information, a feature amount of data Pra′ is generated for the reference model 81. Furthermore, for example, as the input information, a feature amount of data Prb′ is generated for the reference model 82. Moreover, for example, as the input information, a feature amount of data Prc′ is generated for the reference model 83. Thus, the input information that has taken into consideration a range of various parameters in each machine learning model may be generated. In this information processing system 2, a plurality of pieces of the input information generated for each machine learning model are inputted to their corresponding machine learning models, thereby an emotion of the target biological object is determined on the basis of an estimation result obtained from each of the machine learning models. Therefore, an estimation result in accordance with the content and degree of the difference between the arousal baseline of each machine learning model and a user's arousal baseline is obtained from each of the machine learning models. Consequently, even in a case where the arousal baseline assumed at the time of model learning is not consistent with the user's arousal baseline, it is possible to accurately determine an emotion of the target biological object.

Furthermore, in the information processing system 2 according to the present embodiment, a feature amount is acquired on the basis of biological information 10A of the target biological object acquired by using the sensor unit 10 before the biological information 10B used for generation of the input information is acquired. Then, by identifying a plurality of classes of different arousal levels from each other in the acquired feature amount, feature amounts of the plurality of classes are acquired. In this information processing system 2, further, on the basis of the acquired feature amounts of the plurality of classes and feature amounts of a plurality of classes used at the time of learning of one reference model (a first machine learning model) in the plurality of machine learning models, a standardization coefficient of the first machine learning model is converted. Therefore, an estimation result in accordance with the content and degree of the difference between the arousal baseline of each machine learning model and the user's arousal baseline is obtained from each of the machine learning models. Consequently, even in a case where the arousal baseline assumed at the time of model learning is not consistent with the user's arousal baseline, it is possible to accurately determine an emotion of the target biological object.

Moreover, in the information processing system 2 according to the present embodiment, from the feature amounts of a plurality of classes used at the time of learning of one machine learning model (the first machine learning model) in the plurality of machine learning models, a conversion gain (a first conversion gain) with which mapping into the feature amounts of a plurality of classes acquired on the basis of the biological information 10A is performed is derived. Then, by using the derived conversion gain, the standardization coefficient of the first machine learning model is converted. Consequently, even in a case where the arousal baseline assumed at the time of model learning is not consistent with the user's arousal baseline, it is possible to accurately determine an emotion of the target biological object.

Furthermore, in the information processing system 2 according to the present embodiment, by using a second conversion gain that describes a correlation between the feature amounts of a plurality of classes used at the time of learning of the first machine learning model and feature amounts of a plurality of classes used at the time of learning of a second machine learning model different from the first machine learning model and the first conversion gain, a standardization coefficient of the second machine learning model is converted. Consequently, even in a case where the arousal baseline assumed at the time of model learning is not consistent with the user's arousal baseline, it is possible to accurately determine an emotion of the target biological object.

### 4. Modification Example

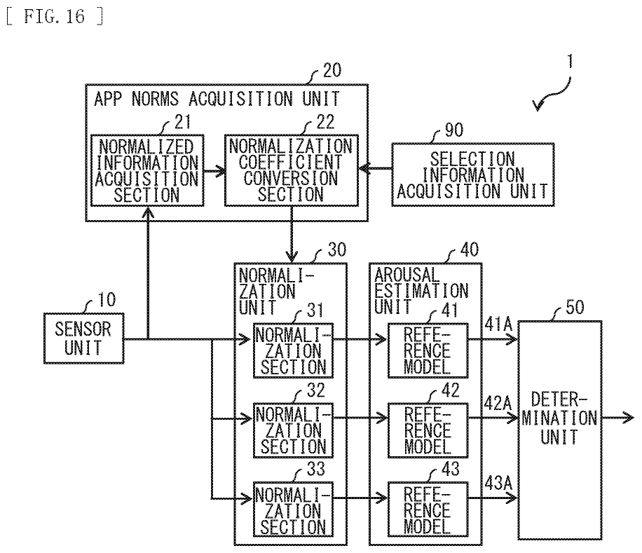

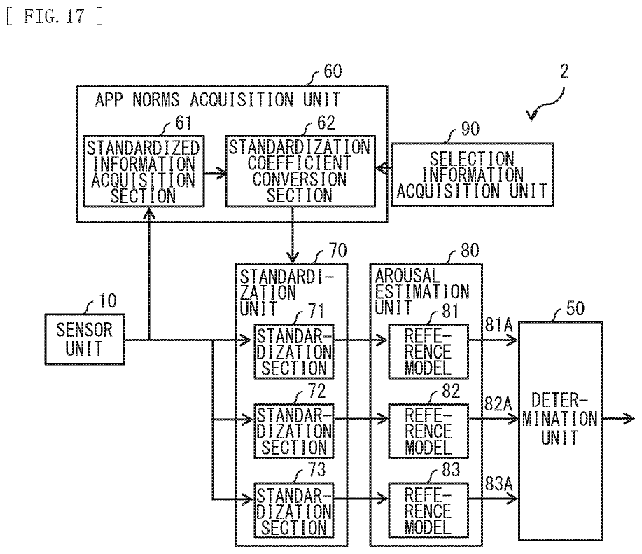

In the respective embodiments described above, the information processing systems 1 and 2 may further include, for example, a selection information acquisition unit 90 as illustrated in FIGS. 16 and 17. The selection information acquisition unit 90 acquires biological information or action information of a target biological object. On the basis of the biological information or behavior information acquired by the selection information acquisition unit 90, the normalization coefficient conversion section 22 and the standardization coefficient conversion section 62 select, from the plurality of machine learning models, a machine learning model having an arousal baseline closest to the arousal baseline of the feature amount X_test obtained on the basis of the biological information 10A.

The selection information acquisition unit 90 includes, for example, a device that acquires, as the biological information, information regarding, for example, brain waves, perspiration, a pulse wave, an electrocardiogram, a blood flow, a skin temperature, a mimetic muscle potential, an electrooculogram, or a specific component contained in saliva. The selection information acquisition unit 90 includes, for example, a device that acquires, as the action information, information regarding, for example, the target biological object's facial expression, voice, blinking, breathing, or the behavioral reaction time.

In the present modification example, by using the selection information acquisition unit 90, a machine learning model having an arousal baseline closest to the arousal baseline of the feature amount X_test obtained on the basis of the biological information 10A is selected from the plurality of machine learning models. This enables the mapping to be accurately performed, and therefore it is possible to accurately determine an emotion of the target biological object.

Furthermore, for example, the present disclosure may have the following configurations.

(1)

An information processing system including:
an input information generation unit that performs predetermined preprocessing on biological information of a target biological object acquired with a sensor, thereby generating input information with respect to each of a plurality of machine learning models; and
a determination unit that determines an arousal of the target biological object on the basis of an estimation result obtained from each of the machine learning models by inputting each of a plurality of pieces of the input information generated for each of the machine learning models to a corresponding one of the machine learning models.
   (2)

The information processing system according to (1), in which
the plurality of machine learning models include different arousal baselines from one another.
   (3)

The information processing system according to (1) or (2), in which
the plurality of machine learning models are generated by using respective pieces of data acquired under environments of different arousal levels from one another.
   (4)

The information processing system according to any one of (1) to (3), in which
the input information generation unit optimizes conditions for the preprocessing in accordance with an action state of the target biological object.
   (5)

The information processing system according to any one of (1) to (4), in which
the preprocessing includes normalization or standardization.
   (6)

The information processing system according to any one of (1) to (5), in which
the machine learning models include a regression model or an identification model.
   (7)

The information processing system according to any one of (1) to (6), further including
the plurality of machine learning models.
   (8)

The information processing system according to (7), in which
each of the machine learning models includes a model generated using normalized information or standardized information of a feature amount obtained on a predetermined arousal baseline and arousal information.
   (9)

The information processing system according to (8), further including:
an acquisition unit that acquires a feature amount on the basis of the biological information of the target biological object acquired from the sensor before acquiring the biological information used for generation of the input information; and
a conversion unit that converts, on the basis of the feature amount acquired by the acquisition unit and a feature amount used at a time of learning of a first machine learning model that is one machine learning model in the plurality of machine learning models, a normalization coefficient or a standardization coefficient of the first machine learning model,
in which the input information generation unit generates the input information with respect to the first machine learning model using the normalization coefficient or the standardization coefficient of the first machine learning model obtained by the conversion unit.
   (10)

The information processing system according to (9), in which
the conversion unit derives, from the feature amount used at the time of learning of the first machine learning model that is one machine learning model in the plurality of machine learning models, a first conversion gain with which mapping into the feature amount acquired by the acquisition unit is performed, and converts the normalization coefficient or the standardization coefficient of the first machine learning model with the derived first conversion gain.
   (11)

The information processing system according to (10), in which
the conversion unit uses a second conversion gain that describes a correlation between the feature amount used at the time of learning of the first machine learning model and the feature amount used at a time of learning of a second machine learning model different from the first machine learning model in the plurality of machine learning models and the first conversion gain to convert a normalization coefficient or a standardization coefficient of the second machine learning model, and
the input information generation unit generates the input information with respect to the second machine learning model using the normalization coefficient or the standardization coefficient of the second machine learning model obtained by the conversion unit.
   (12)

The information processing system according to any one of (1) to (11), further including an information acquisition unit that acquires the biological information or action information of the target biological object,
in which the conversion unit selects, from the plurality of machine learning models, a first machine learning model on the basis of the biological information or the behavior information of the target biological object acquired by the information acquisition unit.

In the information processing system according to one aspect of the present disclosure, the input information corresponding to the biological information of the target biological object acquired with the sensor is generated with respect to each of the plurality of machine learning models. Thus, input information that has taken into consideration a range of various parameters in each machine learning model may be generated. In this information processing system, a plurality of pieces of the input information generated for each machine learning model are inputted to their corresponding machine learning models, thereby the arousal of the target biological object is determined on the basis of an estimation result obtained from each of the machine learning models. Therefore, for example, it is possible to acquire an estimation result in accordance with the content and degree of the difference between the arousal baseline of each machine learning model and a user's arousal baseline from each machine learning model. Consequently, even in a case where the arousal baseline assumed at the time of model learning is not consistent with the user's arousal baseline, it is possible to accurately determine the arousal of the target biological object.

The present application claims the benefit of Japanese Priority Patent Application JP2021-065169 filed with the Japan Patent Office on Apr. 7, 2021, the entire contents of which are incorporated herein by reference.

It should be understood by those skilled in the art that various modifications, combinations, sub-combinations, and alterations may occur depending on design requirements and other factors insofar as they are within the scope of the appended claims or the equivalents thereof.

1. An information processing system comprising:
an input information generation unit that performs predetermined preprocessing on biological information of a target biological object acquired with a sensor, thereby generating input information with respect to each of a plurality of machine learning models; and
a determination unit that determines an arousal of the target biological object on a basis of an estimation result obtained from each of the machine learning models by inputting each of a plurality of pieces of the input information generated for each of the machine learning models to a corresponding one of the machine learning models.

2. The information processing system according to claim 1, wherein the plurality of machine learning models include different arousal baselines from one another.

3. The information processing system according to claim 1, wherein the plurality of machine learning models are generated by using respective pieces of data acquired under environments of different arousal levels from one another.

4. The information processing system according to claim 1, wherein the input information generation unit optimizes conditions for the preprocessing in accordance with an action state of the target biological object.

5. The information processing system according to claim 1, wherein the preprocessing comprises normalization or standardization.

6. The information processing system according to claim 1, wherein the machine learning models include a regression model or an identification model.

7. The information processing system according to claim 5, further comprising the plurality of machine learning models.

8. The information processing system according to claim 7, wherein each of the machine learning models comprises a model generated using normalized information or standardized information of a feature amount obtained on a predetermined arousal baseline and arousal information.

9. The information processing system according to claim 8, further comprising:
an acquisition unit that acquires a feature amount on a basis of the biological information of the target biological object acquired from the sensor before acquiring the biological information used for generation of the input information; and
a conversion unit that converts, on a basis of the feature amount acquired by the acquisition unit and a feature amount used at a time of learning of a first machine learning model that is one machine learning model in the plurality of machine learning models, a normalization coefficient or a standardization coefficient of the first machine learning model, wherein
the input information generation unit generates the input information with respect to the first machine learning model using the normalization coefficient or the standardization coefficient of the first machine learning model obtained by the conversion unit.

10. The information processing system according to claim 9, wherein the conversion unit derives, from the feature amount used at the time of learning of the first machine learning model that is one machine learning model in the plurality of machine learning models, a first conversion gain with which mapping into the feature amount acquired by the acquisition unit is performed, and converts the normalization coefficient or the standardization coefficient of the first machine learning model with the derived first conversion gain.

11. The information processing system according to claim 10, wherein
the conversion unit uses a second conversion gain that describes a correlation between the feature amount used at the time of learning of the first machine learning model and the feature amount used at a time of learning of a second machine learning model different from the first machine learning model in the plurality of machine learning models and the first conversion gain to convert a normalization coefficient or a standardization coefficient of the second machine learning model, and
the input information generation unit generates the input information with respect to the second machine learning model using the normalization coefficient or the standardization coefficient of the second machine learning model obtained by the conversion unit.

12. The information processing system according to claim 1, further comprising an information acquisition unit that acquires the biological information or action information of the target biological object, wherein
the conversion unit selects, from the plurality of machine learning models, a first machine learning model on a basis of the biological information or the behavior information of the target biological object acquired by the information acquisition unit.
